(19) United States
(12) Patent Application Publication
(10) Pub. No.: US 2003/0091647 A1
(43) Pub. Date: May 15, 2003

(54) Controlled dispersion of colloidal suspensions via nanoparticle additions

(76) Inventors: Jennifer A. Lewis, Urbana, IL (US); Valeria Tohver, Andalusia, PA (US); Kenneth Schweizer, Champaign, IL (US); Paul V. Braun, Savoy, IL (US)

Correspondence Address: Brinks, Hofer, Gilson & Lione, NBC Tower, Suite 3600, 455 North Cityfront Plaza, Chicago, IL, 60611, US

(21) Appl. No.: 10/126,941

(22) Filed: Apr. 19, 2002

Related U.S. Application Data
(60) Provisional application No. 60/335,597, filed on Nov. 15, 2001.

(51) Int. Cl. A61K 9/14; A61K 9/16; A61K 9/50

(52) U.S. Cl. 424/490

(57) ABSTRACT

Through the addition of charged nanoparticles to colloidal dispersions of microparticles, the viscosity of the dispersion is modified. By tailoring the potential difference between the microparticles and nanoparticles, the pH, and the amount of nanoparticles added, the phase of the dispersion may be controlled. Through the disclosed methods, colloid flocculation is controlled and colloidal crystals may be isolated.

### REFERENCE TO RELATED APPLICATIONS

This application claims the benefit of U.S. Provisional Application Ser. No. 60/335,597, filed Nov. 15, 2001, entitled “Nanoparticle Engineering of Complex Fluid Behavior,” which is hereby incorporated by reference.

### FEDERALLY SPONSORED RESEARCH OR DEVELOPMENT

This application was funded in part under the following research grants and contracts: NASA Grant No. NAG 8-1471 and NSF DMR 94-53446. The U.S. Government may have rights in this invention.

### BACKGROUND

Colloidal suspensions enjoy widespread use in applications ranging from advanced materials to drug discovery. By tailoring interactions between colloidal particles, one can design stable fluids, gels, and colloidal crystals which may be used in a broad array of applications, including inks, paints, ceramics, coatings, cosmetics, and pharmaceuticals. Colloidal suspensions can also be used to form the precursors or templates for photonic materials that manipulate light in much the same way that a semiconductor manipulates electrons.

Many products are colloid based, including paints, ceramics, and inks. Most any liquid that contains particles that are not fully solubilized can be characterized as a colloidal suspension. The viscosity of colloid dispersions can vary over a wide range from liquid to gel. Additionally, when the suspended particles slowly settle from the colloidal dispersion, they may settle in a very ordered or “crystalline” fashion. Colloids may be useful not only in their native state, such as paints, but to form highly ordered solids which are then turned into photonic materials. In the future, such photonic materials may play an important role in optical communication and computing technologies.

### BRIEF SUMMARY

The viscosities of colloidal dispersions are modified by adding charged nanoparticles to microparticle dispersions. The zeta potential difference between the microparticles and the nanoparticles is at least ten millivolts.

Colloidal dispersions are provided that demonstrate an increased resistance to flocculation. Flocculation resistance is provided through the addition of charged nanoparticles to the colloidal dispersions.

A method of changing the phase of a colloidal dispersion from a gel, to a fluid, and back to a gel through the increasing addition of charged nanoparticles is provided.

The scope of the present invention is defined solely by the appended claims, and is not affected to any degree by the statements within this summary.

### BRIEF DESCRIPTION OF THE DRAWINGS

FIG. 1 is a plot showing an increase in zeta potential (effective charge) of the colloidal microparticles with increasing nanoparticle volume fraction addition at pH=1.5.

FIG. 2 is a plot showing the quantity of nanoparticles that associate with microparticles as a function of nanoparticle concentration in the carrier liquid at pH=1.5.

FIG. 3 is a plot of nanoparticle adsorption onto an oxidized silicon wafer over time at pH=1.5.

FIG. 4 is a plot of nanoparticle adsorption onto an oxidized silicon wafer over time at pH=4.0.

FIG. 5 and 6 show the phase behavior of microparticle/nanoparticle mixtures as the nanoparticle volume fraction is increased in relation to the microparticle volume fraction.

FIG. 7 is a two-dimensional image obtained by confocal microscopy of a colloidal crystal formed by allowing gravity-settling to occur of a nanoparticle stabilized fluid-phase colloid.

FIG. 8 shows the average center-to-center separation distance in nanometers between microparticles that have settled from a colloidal dispersion as the depth of the settled microparticles increases (solid squares).

FIG. 9 is a viscous response plot of apparent viscosity as a function of shear rate for microparticle/charged nanoparticle dispersions at varying microsphere volume fraction (φnano).

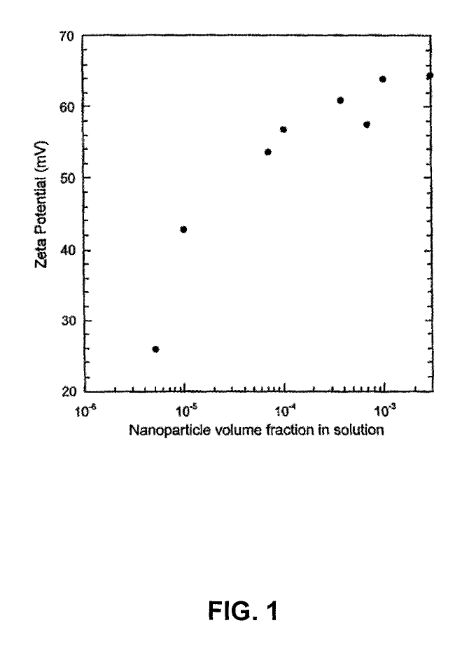

### DETAILED DESCRIPTION

The present invention includes stabilized colloids and methods for imparting stability to colloidal dispersions. By controlling colloidal stability, the structure and properties of the colloids, i.e. viscosity, may be altered by several orders of magnitude. The current invention may be applied to most technologies involving particulate suspensions. Applicable technologies include ceramics, ceramic substrates for electronic packaging, capacitors, mesoporous structures, photonics, inks, paints, coatings, cosmetics, food products, drilling muds, dyestuffs, foams, agricultural chemicals, and pharmaceuticals.

In addition to forming colloidal suspensions for direct use, the present invention may be used to form periodic or crystalline materials from colloidal suspensions. By stabilizing the colloid through nanoparticle addition to form a fluid phase and allowing the colloidal particles to slowly settle from the carrier liquid, a crystalline colloidal phase results. When the carrier liquid is removed, this crystalline phase can then be used directly or as a template.

If appropriate microparticles are chosen, the crystalline material may be solidified to directly form solid structures, such as ceramic substrates for electronic devices, or to form electronic materials, such as would be suitable for use in capacitors. The crystalline material may be solidified using heat or other methods which bring about the desired solidification.

A liquid containing a photonic material may also be added to the crystalline template to form a surrounding matrix. When the colloidal template is then removed from the matrix, porous materials can be created that are strong and have a suitably high refractive index for photonic applications. Preferably, a refractive index of greater than 3 is obtained for these materials. Such colloidal derived crystals can be used in various applications including band gap optical switching and lithographic applications. See Braun, et al., Nature, Vol. 402, pp. 603-04 (1999) and Braun, et al., Europhys. Lett. 56, (2), pp. 207-13 (2001) for a more complete discussion of photonic applications.

Many pharmaceutical uses also exist for a colloidal suspension having adjustable viscosity. Because the amount of colloidal stabilization provided by a specific type and quantity of nanoparticle is pH dependent, pH changes that occur when the colloidal suspension is administered orally, subcutaneously, or intravenously can be used to alter the phase of the nanoparticle stabilized colloid. One such use, for example, is to alter the viscosity of injectable pharmaceutical compositions containing one or more bioactive agent.

It can be advantageous to have a very low viscosity drug composition that can pass through a very fine needle into the body, but yet have the drug composition stay localized in the tissue at the region of injection. This localized region is characterized by its phase separation from the physiological fluid and its decreased fluidity relative to the original suspension. By adding charged nanoparticles to the colloidal drug composition that lower viscosity at the pH of the delivery suspension, but do not at physiological pH, a drug composition can exist at a relatively low viscosity in the syringe, but at a relatively higher viscosity in the body. In this fashion, the pharmaceutical is easily delivered to a specific tissue location.

Bioactive agents, which may be delivered by colloidal suspensions, include drugs that act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, anti-inflammatory corticosteroids, ocular drugs and synthetic analogs of these species.

Examples of drugs which may be delivered by colloidal suspensions include, but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and des-phenylalanine human growth hormone, bovine growth hormone and porcine growth hormone, fertility inhibitors such as the prostaglandins, fertility promoters, growth factors such as insulin-like growth factor, coagulation factors, human pancreas hormone releasing factor, analogs and derivatives of these compounds, and pharmaceutically acceptable salts of these compounds, or their analogs or derivatives.

Other bioactive agents, which may be delivered by colloidal suspensions, include chemotherapeutic agents, such as carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986.

Not only can many of these bioactive agents, including proteins, be formed directly into colloidal suspensions, but they can also be mixed with biodegradable compositions or polymers to form microparticles. By grinding a mixture containing one or more biodegradable composition and bioactive agent into microparticles, colloidal dispersions may be formed with the present invention. Many useful biodegradable compositions suitable for use with bioactive agents may be found in U.S. Pat. No. 5,416,071.

Examples of useful biodegradable polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof. Methods of forming microparticles from mixtures containing bioactive agents and biodegradable polymers are disclosed in EPO 0 263 490.

Colloidal Dispersions

Colloidal particles or microparticles have a substantial fraction of their atoms or molecules at the surface. While not necessary, these microparticles may often be hollow. When placed in a carrier liquid, an interface exists between the surface of the microparticles and the carrier liquid. The behavior of the resultant colloid, including stability, digestibility, film forming properties, and viscous and elastic properties, is chiefly determined by how this surrounding interface interacts with the surface of the colloidal particles and the carrier liquid.

Solutions, unlike colloidal dispersions or suspensions, lack an identifiable interface between their solubilized molecules and the solvent. In solutions, the solubilized molecules are in direct contact with the solvent, while in colloidal dispersions only the surface of the microparticles are in direct contact with the carrier liquid. Hence, the carrier liquid does not solubilize the particles that make up a colloid; instead, the carrier liquid “carries” the microparticles. By carrying the microparticles, a suspension or dispersion results. The terms suspension and dispersion are used interchangeably.

The interfaces between the suspended colloidal microparticles, and the carrier liquid or liquid mixture in which they reside, play the dominant role in determining the behavior and capabilities of the colloidal dispersion. Colloidal dispersions are considered stable if the particles that form the colloid are separated or deflocculated, i.e., not aggregated or flocculated. In general, the term stability in relation to colloidal dispersions relates to the dispersion's resistance to change over time.

Long-range attractive forces, such as van der Waals forces, are believed to pull colloidal particles together. When colloidal particles are pulled together, the colloidal dispersion or suspension is destabilized. This destabilization is often referred to as aggregation or flocculation and can result in precipitation of the aggregated particles from the colloidal dispersion.

Alternatively, columbic, steric, and other repulsive interactions are believed to repel colloidal particles from each other. If the particles cannot aggregate together, the stability of the colloidal dispersion is increased and flocculation may be reduced.

A traditional view is that the addition of small particles or other species destabilize colloidal dispersions. By destabilizing the dispersion through the addition of small particles, flocculation or aggregation is increased. While the ability to flocculate colloidal particles and remove them from the liquid carrier may be advantageous in some instances, such as the removal of impurities during water purification, it is disadvantageous when a process requires the particles remain in suspension. Hence, it is desirable to exercise control over the stability of the colloidal dispersion.

Surprisingly, the claimed invention provides embodiments that can reduce the tendency of the particles present in a colloidal dispersion (microparticles) to aggregate or flocculate through the addition of charged nanoparticles. Thus, the microparticles are stabilized against flocculation. Even if the particles begin to settle from the carrier liquid, they tend to settle as individual particles, not as larger aggregates.

By altering the charge, nature, and quantity of nanoparticles added to the colloidal dispersion, the present embodiments allow for the stability of the colloidal suspension to be increased or decreased. While not wishing to be bound by any particular theory, it is believed that the charged nanoparticles stabilize the colloidal dispersion by increasing the coulombic repulsion between the microparticles.

One possible explanation is that the like-charged nanoparticles congregate about the microparticles, thus forming a charged “halo” about the microparticles. Because the nanoparticles carry the same charge, the microparticles repel each other. The repulsive forces generated by the halos reduce the tendency of the particles to aggregate, thus counteracting the attractive van der Wall's forces. It does not matter if the charge carried by the nanoparticles is positive or negative.

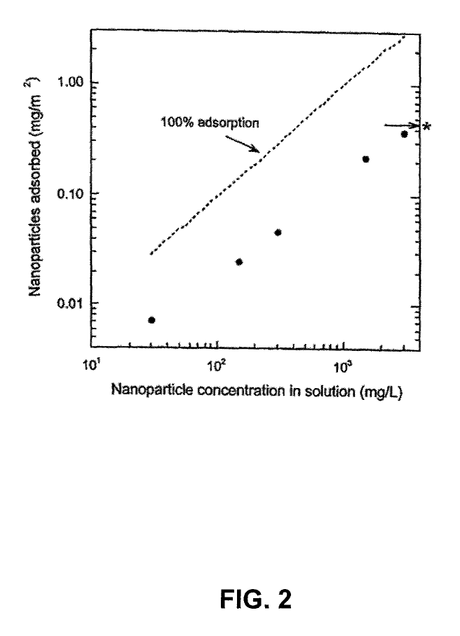

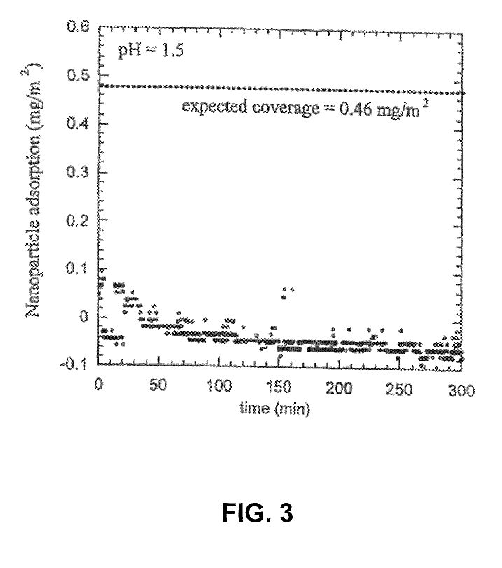

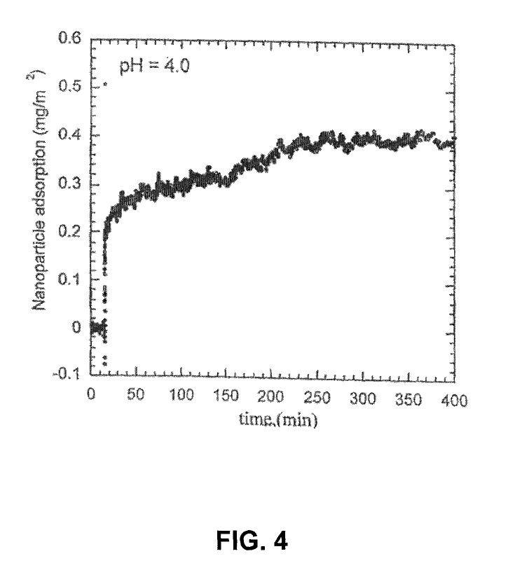

FIGS. 2, 3, and 4 support this explanation. In FIG. 2 the distance of the data points from the 100% adsorption line suggests that the nanoparticles are not strongly adsorbed onto the microparticle surfaces, but that they loosely associate with the microparticles. In FIG. 3, the distance of the data points from the expected coverage dashed line suggests that the nanoparticles associate with the surface, but are not adsorbed onto it. There is essentially no build up of nanoparticles on the wafer. FIG. 4 shows that if the pH is increased from 1.5 to 4.0 the nanoparticles can be driven onto the surface of the wafer due to their opposite charge at this pH. The plot suggests that at pH=1.5 the nanoparticles are not adsorbed onto the microparticles.

Microparticles

The microparticles are any particle that can be suspended or dispersed in a carrier liquid to form a colloidal suspension. While the composition of the microparticles is not important, preferable microparticles include metals, polymers, ceramics, semiconductors, bioactive agents, proteins, liposomes, and other biomolecules. Depending on their surface structure and the nature of the carrier liquid, the effective particle diameter of microparticles suitable for colloid formation can vary over a wide range. By “effective particle diameter” it is meant the longest dimension of the particle. Thus, if a particle is 0.01 μm in one dimension and 10 μm in another, the effective diameter of the particle is 10 μm.

Preferred microparticles have effective particle diameters of 0.01 μm to 100 μm, more preferably from 0.05 μm to 10 μm, and most preferably 0.2 μm to 3 μm.

Nanoparticles

Altering the quantity, nature, and charge of nanoparticles added to the colloidal dispersion changes the stability of the dispersion. The nanoparticles may be added to a colloidal suspension already prepared, or the microparticles may be added to a suspension containing the nanoparticles.

The preferred quantity of nanoparticles which must be added to a particular colloidal dispersion to yield the desired phase is dependent on the nature of the microparticles, the polarity of the carrier liquid, and the charge carried by the nanoparticles at the pH of interest. In addition to the quantity of nanoparticle addition, the type of nanoparticle may also be changed to produce less or more stabilization at similar volume amounts depending on the nature of the colloidal particles and the carrier liquid.

Preferred nanoparticles are any particle that naturally has, or can be functionalized to adopt, a surface charge in a polar liquid. Preferred naturally charged nanoparticles include those made from metal oxides or nitrides. Examples of preferred particles that naturally adopt a surface charge in polar liquids include zirconium oxide, aluminum oxide, silicon dioxide, titanium dioxide, and silicon carbide.

Examples of preferred particles that can be functionalized to adopt a surface charge in a polar liquid include those made from polymers, semi-conductors, and metals. Preferred polymer nanoparticles include those made from polymethyl methacrylate, polystyrene, polylactic acids, and acrylic latexes. Preferred semi-conductor nanoparticles include those made from silicon and germanium. Preferred metal nanoparticles include those made from gold and silver. Any of these nanoparticles may be preferably functionalized with carboxylic acids, amines, sulfates, or other functional groups that allow them to carry a charge. Preferable functional groups include those that allow the nanoparticles to carry a positive charge, such as amines, or functional groups that allow the nanoparticles to carry a negative charge, such as carboxylic acids.

Preferred nanoparticles have an effective particle diameter of at most 33,000 nm, more preferably from 1 nm to 3,300 nm, and most preferably from 1 nm to 330 nm. The ratio of the effective diameter of nanoparticles to the effective diameter of the microparticles is preferably at least 1 to 3, more preferably at least 1 to 6, and most preferably at least 1 to 10.

Preferably, the zeta potential difference between the charge carried by the nanoparticles and the microparticles is at least 10 millivolts, more preferably at least 25 millivolts, and most preferably at least 60 millivolts.

Effective Charge

The effective charge carried by the nanoparticles varies with the pH of the suspension and may be determined by electrophoresis and other methods. The isoelectric point is the pH value where the particles have no charge in the selected polar liquid and can be tuned through nanoparticle selection. While multiple methods exist to determine the effective charge of a nanoparticle, one way is to measure the nanoparticle's zeta potential by electrophoresis. A common instrument used for this determination is a ZETASIZER, available from Malvern Instruments, Southborough, Mass., USA.

In general, zeta potential is determined by preparing a very dilute, approximately 1 part-per-million sample of nanoparticles in a liquid carrier. A small amount of the dilute sample is transferred to a sample cell that is placed in the instrument between two electrodes. An electric field is then applied between the electrodes, which causes any charged nanoparticles to migrate through the liquid carrier. A pair of laser beams is used to measure the velocity of the migrating nanoparticles. Because the electric potential applied to the plates and the velocity of the nanoparticles is known, the effective charge or zeta potential of the charged nanoparticles may be calculated.

Stabilized Against Flocculation

A colloidal dispersion is stabilized against flocculation when at least 90% of the microparticles can be observed as being individual, rather than aggregated in groups of two or more. This determination is made by diluting a sample of the dispersion to 1 part-per-million solids, placing the sample on a slide, and observing by light microscopy.

Carrier Liquid

A feature of the present approach to colloidal phase control is that the nanoparticles are highly charged in relation to the charge present on the colloidal microparticles. A closely related consideration is the polarity of the carrier liquid in which the colloidal particles are dispersed. The carrier liquid must have sufficient polarity to support the charged nanoparticles and the microparticles.

While the colloid dispersions of the present embodiments are not solubilized, the microparticles and nanoparticles are suspended or dispersed in a carrier liquid. Depending on the charge of the nanoparticles used, varying degrees of carrier liquid polarity may be required to keep the nanoparticles suspended. Depending on the effective charge of the nanoparticles and the nature of the microparticles, mixtures of polar liquids and less-polar, or even non-polar liquids can be used to fine tune the polarity of the liquid carrier.

While many polar carrier liquids may be used to form the colloid dispersions, water is the most preferred carrier. Other preferred carrier liquids include alcohols, such as methanol, propanol, ethanol, and t-butanol, N,N-dimethylformamide (DMF), dimethyl sulfoxide (DMSO), acetone, acetonitrile, acetic acid, hexamethylphosphoric triamide (HMPA), tetrahydrofuran (THF), N,N-dimethylacetamide, N-methyl-2-pyrrolidone, tetramethyl urea, glycerol, and ethylene glycol, or mixtures thereof.

Phase Transitions

In the preferred embodiments, colloidal dispersions may be produced that undergo phase transitions between fluid, gel, crystalline, and glassy states. A crystalline state is determined when long range order is observed by diffraction, such as light diffraction. A glassy state is present when the motion of the particles appears to cease, as observed by confocal microscopy.

Colloidal dispersions undergo a transition from a colloidal gel to a stable fluid, to a flocculated colloidal gel as the volume of nanoparticle addition is increased. System stability is reversed at higher nanoparticle volume fractions where a fluid to gel transition is produced. If additional nanoparticles are added, the microparticles will aggregate and settle from the carrier liquid.

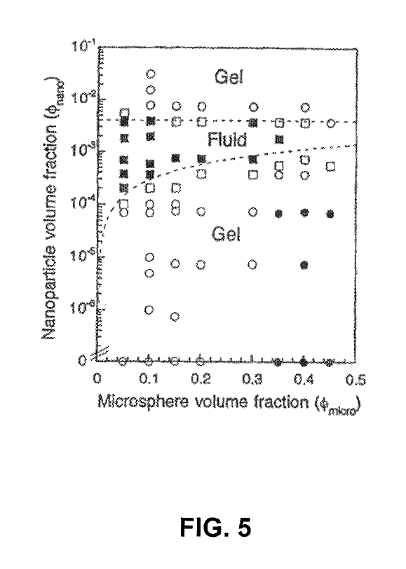

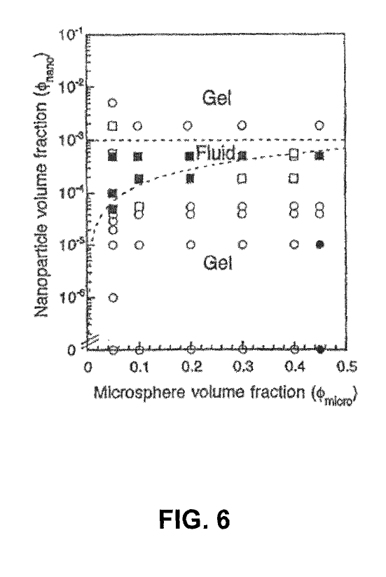

FIGS. 5 and 6 show the phase behavior of microparticle/nanoparticle mixtures as the nanoparticle volume fraction is increased in relation to the microparticle volume fraction. Each circle or square represents a different prepared sample. Open circles represent samples containing a mixture of a weak colloidal gel with a nanoparticle fluid. Filled circles represent samples containing a mixture of colloidal gel and nanoparticle fluid. Filled squares represent samples containing a homogenous fluid of colloidal microparticles and nanoparticles. Open squares represent samples that have separated into a homogenous fluid of colloidal microparticles and nanoparticles and a weak colloidal gel. The lower and upper dashed lines represent the required nanoparticle volume fraction to affect the gel to fluid and fluid to gel transitions, respectively.

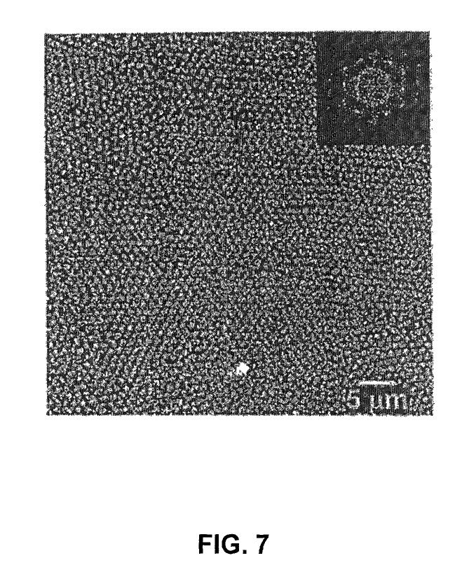

If the microparticles from the stabilized fluid phase are allowed to gravity-settle, an ordered, or crystalline phase forms. Thus, at lower nanoparticle volume addition, the dispersion is stabilized from a gel to a liquid phase, and at higher nanoparticle volume fractions, colloidal stability is reversed, driving the liquid phase to a flocculated gel phase. The highly ordered or periodic nature of the microparticles and voids within the crystalline sediment is evident from FIG. 7.

There is no specific point at which a gel becomes a liquid. In general, however, liquids freely flow while gels do not. A liquid will conform to the shape of a container in which it is placed, while a gel can have a physical form separate from the container where it resides.

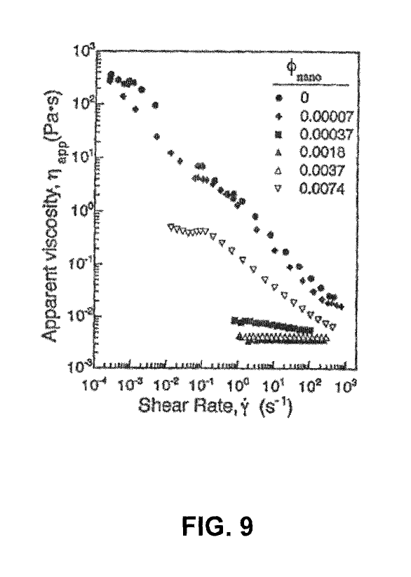

FIG. 9 is a viscous response plot of apparent viscosity as a function of shear rate for microparticle/charged nanoparticle dispersions at varying microsphere volume fraction (φnano). As nanoparticle concentration increases, the apparent viscosity of the colloid in relation to shear rate decreases. An approximate six order of magnitude decrease in low shear viscosity is observed with increasing nanoparticle addition. At the φnano=0.0074 concentration, a reversal of the trend is observed.

When sufficient nanoparticles are added to the colloidal dispersion to form the stabilized fluid, but prior to aggregation, the microparticles will eventually gravity-settle from the carrier liquid and form an ordered network or crystalline phase. While not wishing to be bound by any particular theory, it is believed that the nanoparticles reside in the interstices formed by the settled microparticles, thus providing some stabilization to the structure. These crystalline phases are robust enough that the carrier liquid may be removed without collapse.

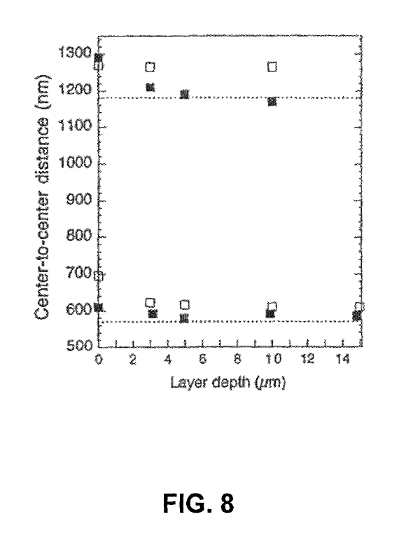

FIG. 8 shows the average center-to-center separation distance in nanometers between microparticles that have gravity-settled from a colloidal dispersion as the depth of the settled microparticles increases (solid squares). This crystalline settled phase or sediment resulted from a microparticle/charged nanoparticle dispersion having a zeta potential (effective charge) of 60 mV. The open squares represent a settled phase created by allowing microparticles to settle at a 60 mV effective charge brought about by pH change alone. While in both instances the microparticles were allowed to settle at similar effective charges, the further departure from the dashed lines by the samples without the nanoparticles establish that packing efficiency is reduced. Thus, the addition of charged nanoparticles results in higher ordered settled crystalline phases that have physical contact between the individual microparticles. As layer depth increases, the distance between microparticles is also reduced.

The preceding description is not intended to limit the scope of the invention to the preferred embodiments described, but rather to enable any person skilled in the art of colloidal suspensions to make and use the invention.

### EXAMPLES

Example 1: Determining effective charge through a zeta potential.

0.45 g of silica microparticles (a_{SiO2}=0.285 μm), available from Geltech (Orlando, Fla.), were dispersed in 19.76 mL deionized water while stirring (φ_{SiO2}=0.01). The resultant silica suspension was ultrasonicated with 1 sec pulse-on/off for 5 min followed by rigorous stirring for 2 hrs. The suspension was then re-ultrasonicated for 5 min and acidified to pH=1.5 with 0.04 mL of concentrated nitric acid (HNO₃).

A binary mixture of φ_{SiO2}=1×10⁻⁴ and φ_{ZrO2}=1×10⁻¹ was prepared as follows: 0.6 mL of the silica microparticle suspension (®_{SiO2}=0.01) was diluted with 29.4 mL of a pH=1.5 nitric acid/deionized water solution to a final volume of 30 mL with φ_{SiO2}=2×10⁻⁴. The diluted silica suspension was then ultrasonicated for 1 min.

A nitrate-stabilized zirconia solution of φ_{ZrO2}=2×10⁻³ was prepared by mixing 0.405 mL of zirconia solution (φ_{ZrO2}=7.4×10⁻²/ Zr 10/20 as-received from Nyacol Nano Technologies; Ashland, Mass.) with a solution of pH=1.5 nitric acid/deionized water to a final volume of 30 mL. This zirconia solution was added to the above dilute silica microparticle suspension. The resultant binary mixture containing silica microparticles and zirconia was then ultrasonicated for 5 minutes and stirred for an additional 4 hrs. Zeta-potential measurements of the mixture were then taken by microelectrophoresis in a Laser Zee Model 501 (Pen Kem; Bedford Hills, N.Y.). Silica-zirconia binary mixtures with different volume fractions of zirconia were obtained by varying the amount of zirconia in the 30 mL suspensions.

Example 2: Synthesis of a colloidal dispersion in the gel phase.

12.15 g of silica microparticles (a_{SiO2}=0.285 μm) were dispersed in 39.5 mL of deionized water while stirring (φ_{SiO2}=0.12). The resultant suspension was ultrasonicated with 1 sec on/off pulses for 5 min, followed by rigorous stirring for 2 hrs. Ultrasonication and stirring were repeated three times to fully disperse the suspension. After overnight stirring, the suspension was re-ultrasonicated for 5 min and adjusted to pH=1.5 with 0.103 mL of concentrated nitric acid (HNO₃).

In order to obtain a colloidal gel, a final binary mixture volume of 15 mL was prepared as follows. For a binary mixture with φ_{SiO2}=0.1 and φ_{ZrO2}=8.24×10⁻⁵, 12.479 mL of silica suspension was first diluted with 2.505 mL of pH=1.5 nitric acid/deionized water and mixed with 0.0167 mL of zirconia solution, as-received from Nyacol Nano Technologies (Ashland, Mass.). The mixture was ultrasonicated for 1 min and rigorously stirred for an additional 4 hrs. Then, 10 mL of the mixture was transferred to a graduated cylinder that served as a sedimentation column. The particles consolidated by gravity-driven sedimentation for 1-2 weeks. Colloidal gels were obtained from binary mixture suspensions with φ_{SiO2}=0.1 and φ_{ZrO2} from 0 to 3×10⁻⁴.

Example 3: Conversion of a colloidal gel to a colloidal fluid through the addition of charged nanoparticles.

A colloidal fluid was prepared in a similar fashion to the colloidal gel from Example 2, with the exception that 0.167 mL of as-received zirconia suspension (Nyacol Nano Technologies; Ashland, Mass.) was added to obtain φ_{ZrO2}=8.24×10⁻⁴. Colloidal fluid regimes were observed for suspensions with φ_{SiO2}=0.1 and φ_{ZrO2} between 3×10⁻⁴ and 4×10⁻³.

Example 4: Conversion of a colloidal fluid to a colloidal flocculated gel through the continued addition of charged nanoparticles.

The pH of the colloidal fluid from Example 3 was re-adjusted to pH=1.5 with ammonium hydroxide (NH₄OH) and additional zirconia suspension was added. Colloidal re-gelation regimes were observed for suspensions with φ_{SiO2}=0.1 and φ_{ZrO2} greater than about 4×10⁻³.

Prophetic Example 1: Synthesis and isolation of a colloidal crystal from a colloidal fluid.

The binary colloidal fluid (from Example 3) can give rise to a colloidal crystalline phase under gravity-driven consolidation. The crystal is isolated after pipetting away the excess aqueous solution followed by slowly drying the remaining consolidated colloid. The crystallinity of the resultant colloid is analyzed with confocal microscopy. When microparticles having an approximate diameter of 0.5-1 μm are used to form the colloid, the resultant colloidal crystal is opalescent.

Prophetic Example 2: Conversion of a non-charge bearing nanoparticle to a nanoparticle capable of bearing a charge through functionalization.

Sulfate functionalized polystyrene nanoparticles can be synthesized by surfactant-free emulsion polymerization. The polymerization for sulfate-functionalized polystyrene (˜100 nm) can be carried out as follows: 965 mL of deionized water and 0.4505 g of styrene monomer (Sigma-Aldrich; Milwaukee, Wis.) are charged in the round-bottomed flask while stirring and purging with nitrogen gas. This mixture is refluxed at 60° C. for 30 min. The reaction is started by injecting 1.5 g of potassium peroxodisulfate initiator (Sigma-Aldrich; Milwaukee, Wis.) dissolved in 35 mL of degassed water and followed by re-heating to 60° C. after injecting the initiator solution within less than 1 min. After 4 hrs, when the turbidity levels off at a constant value, the polymerization is stopped. The latex is dialysed against distilled water over a period of 4 weeks. During this time, the distilled water is replaced twice every 24 hrs. Amine-functionalized polystyrene can be similarly synthesized using 2,2′-azobis(2-amidinopropane)-dihydrochloride (Wako chemicals; Richmond, Va.) as an initiator.

As any person skilled in the art of colloidal suspensions will recognize from the previous description, FIGS., and examples that modifications and changes can be made to the preferred embodiments of the invention without departing from the scope of the invention defined by the following claims.

What is claimed:

1. A method of forming a colloidal dispersion, comprising:
nanoparticles and microparticles,
wherein said nanoparticles carry a charge, and
a zeta potential difference between said microparticles and said nanoparticles is at least 10 millivolts.

2. The method of claim 1, wherein the microparticles in said colloidal dispersion are stabilized against flocculation.

3. The method of claim 1, wherein the zeta potential difference between said microparticles and said nanoparticles is at least 25 millivolts.

4. The method of claim 1, wherein the zeta potential difference between said microparticles and said nanoparticles is at least 60 millivolts.

5. The method of claim 1, wherein the ratio of the effective diameter of the nanoparticles to the effective diameter of the microparticles is at least 1 to 3.

6. The method of claim 1, wherein the ratio of the effective diameter of the nanoparticles to the effective diameter of the microparticles is at least 1 to 6.

7. The method of claim 1, wherein the ratio of the effective diameter of the nanoparticles to the effective diameter of the microparticles is at least 1 to 10.

8. The method of claim 1, wherein said colloidal dispersion comprises water.

9. The method of claim 8, wherein said colloidal dispersion further comprises a liquid less polar than water.

10. The method of claim 9, wherein said liquid is selected from the group consisting of alcohol, methanol, propanol, ethanol, t-butanol, N,N-dimethylformamide, dimethyl sulfoxide, acetone, acetonitrile, acetic acid, hexamethylphosphoric triamide, tetrahydrofuran, N,N-dimethylacetamide, N-methyl-2-pyrrolidone, tetramethyl urea, glycerol, and ethylene glycol, or mixtures thereof.

11. The method of claim 1, wherein said nanoparticles have an effective diameter of at most 33,000 nm.

12. The method of claim 1, wherein said nanoparticles have an effective diameter from 1 nm to 330 nm.

13. The method of claim 1, wherein said microparticles have an effective diameter from 0.01 μm to 100 μm.

14. The method of claim 1, wherein said microparticles have an effective diameter from 0.2 μm to 3 μm.

15. In a colloidal dispersion including microparticles and a carrier liquid, the improvement comprising increasing the stabilization of said microparticles against flocculation by the presence of nanoparticles, wherein said nanoparticles carry a charge having a zeta potential difference from said microparticles of at least 10 millivolts.

16. The colloidal dispersion of claim 15, wherein said zeta potential difference is at least 60 millivolts.

17. A colloidal dispersion comprising:
microparticles;
a carrier liquid; and
nanoparticles, wherein said nanoparticles carry a charge having a zeta potential difference from said microparticles of at least 10 millivolts.

18. The colloidal dispersion of claim 17, wherein the ratio of the effective diameter of the nanoparticles to the effective diameter of the microparticles is at least 1 to 3.

19. The colloidal dispersion of claim 17, wherein the ratio of the effective diameter of the nanoparticles to the effective diameter of the microparticles is at least 1 to 6.

20. The colloidal dispersion of claim 17, wherein the ratio of the effective diameter of the nanoparticles to the effective diameter of the microparticles is at least 1 to 10.

21. The colloidal dispersion of claim 17, wherein the zeta potential difference between said microparticles and said nanoparticles is at least 25 millivolts.

22. The colloidal dispersion of claim 17, wherein the zeta potential difference between said microparticles and said nanoparticles is at least 60 millivolts.

23. The colloidal dispersion of claim 17, wherein said nanoparticles have an effective diameter of at most 33,000 nm.

24. The colloidal dispersion of claim 17, wherein said nanoparticles have an effective diameter from 1 nm to 330 nm.

25. The colloidal dispersion of claim 17, wherein said microparticles have an effective diameter from 0.01 μm to 100 μm.

26. The colloidal dispersion of claim 17, wherein said microparticles have an effective diameter from 0.2 μm to 3 μm.

27. An ink comprising the colloidal dispersion of claim 17.

28. A method of making the ink of claim 27, comprising:
adding nanoparticles to a colloidal dispersion.

29. A pharmaceutical composition comprising the colloidal dispersion of claim 17.

30. A method of making the pharmaceutical composition of claim 29, comprising:
adding nanoparticles to a colloidal dispersion.

31. A periodic material comprising the colloidal dispersion of claim 17, wherein said microparticles are in a crystalline state.

32. A method of making a photonic material, comprising:
providing the periodic material of claim 31;
removing at least a portion of said carrier liquid from the periodic material to form a crystalline sediment; and
adding a liquid comprising a photonic material to said crystalline sediment which solidifies to form a surrounding matrix, wherein said matrix has a refractive index of greater than 3.

33. A method of making a ceramic substrate, comprising:
providing the periodic material of claim 31;
removing at least a portion of said carrier liquid from the periodic material to form a crystalline sediment; and
solidifying said crystalline sediment to form said ceramic substrate.

34. A capacitor, comprising the colloidal dispersion of claim 17.

35. A method of making a capacitor, comprising:
providing the periodic material of claim 31;
removing at least a portion of said carrier liquid from the periodic material to form a crystalline sediment; and
solidifying said crystalline sediment to form said capacitor.

36. A method of changing the phase of a colloidal dispersion from a gel phase to a liquid phase, comprising:
adding nanoparticles to the dispersion, to form a mixture,
wherein said nanoparticles in said mixture carry a charge resulting in a zeta potential difference between said microparticles and said nanoparticles of at least 10 millivolts.

37. A method of changing the phase of a colloidal dispersion from a liquid phase to a gel phase, comprising:
adding nanoparticles to the dispersion, to form a mixture,
wherein said nanoparticles in said mixture carry a charge resulting in a zeta potential difference between said microparticles and said nanoparticles of at least 10 millivolts.

38. A method of changing the phase of a colloidal dispersion from a gel phase to liquid phase to a gel phase, comprising:
adding nanoparticles to the dispersion, to form a mixture, wherein said charged nanoparticles carry a charge resulting in a zeta potential difference between said microparticles and said nanoparticles of at least 10 millivolts.
